(19) United States
(12) Patent Application Publication
(10) Pub. No.: US 2012/0245466 A1
(43) Pub. Date: Sep. 27, 2012

(54) METHOD AND SYSTEM FOR CAPTURING MEDICAL IMAGING DATA

(75) Inventor: Somnath GANGULY, Banagalore (IN)

(73) Assignee: INFOSYS TECHNOLOGIES, LTD., Bangalore (IN)

(21) Appl. No.: 13/311,811

(22) Filed: Dec. 6, 2011

(30) Foreign Application Priority Data
Mar. 25, 2011 (IN) 934/CHE/2011

(51) Int. Cl. A61B 8/13; A61B 6/00

(52) U.S. Cl. 600/443; 600/407

(57) ABSTRACT

A portable medical image capturing apparatus is disclosed. The apparatus includes a plurality of transducers that are enabled to capture medical imaging data of a patient. The plurality of transducers are placed on an enclosure. The plurality of transducers capture the medical imaging data when the enclosure is held close to the patient. Further, the apparatus includes a communications module to transmit the medical imaging data of the patient to a remote data processing unit. The remote data processing unit is enabled to enhance the captured medical imaging data of the patient.

This application claims the benefit of Indian Patent Application Filing No. 934/CHE/2011, filed Mar. 25, 2011, which is hereby incorporated by reference in its entirety

### FIELD OF THE INVENTION

The present invention generally relates to the field of medical imaging systems, and in particular to a method and system for capturing medical imaging data.

### BACKGROUND

Traditional ultrasound systems are large and bulky in nature. The size of these ultrasound systems present in hospitals do not permit portability and hence make them tied to a place or a laboratory. The lack of movement of such facilities makes it difficult to treat patients in an emergency situation. Modern ambulances and remote hospitals provide for most life support facilities. However, patients lack immediate care in some cases due to the lack of availability of devices that can monitor internal systems of the body. Some patients are also advised rest at home and they need to be kept under constant watch. It becomes cumbersome for such patients to travel to the hospital for regular ultrasound tests. Hence it is desirable to have a system that facilitates capturing ultrasound data through a device that is located in physical proximity of the patient.

In many emergency cases, help for a patient is important immediately. The doctors may require good medical images of the internal systems of the patient to deal with the problem. In such cases, it would be desirable to have a system that allows the doctor to collect medical imaging data from a handheld device and get the low-quality data processed at a remote data center for further usage.

Some systems have been developed to facilitate capturing ultrasound medical data of a patient through a handheld device. The device helps is capturing imaging data after it is placed on the patient's body. These systems involve displaying the medical imaging data of the patient on a display device that is coupled to the handheld probe using a wired connection.

The system thus makes it easy for the ultrasound tests to be conducted on patients located in a limited range.

Further, the medical imaging data that is captured through these probes is high quality data and thus the file size is huge. Present systems do not allow a doctor to capture imaging data of the user at a lesser resolution and then convert or enhance it to one of higher resolution.

Hence, there is a need for a method and a system that captures medical imaging data through a device located in physical proximity of the user and transfers it for conversion to a higher resolution.

### SUMMARY

An embodiment of the present invention provides for a portable medical image capturing apparatus. The portable medical image capturing apparatus includes a plurality of transducers that are enabled to capture medical imaging data of a patient. The plurality of transducers, according to one embodiment, are placed on an enclosure and are enabled to capture imaging data when the enclosure is placed near the patient. Further, the apparatus includes a communications module that is enabled to transmit the medical imaging data of the patient to a remote data processing unit. The remote data processing unit enhances the image captured by the plurality of transducers.

Another embodiment of the present invention provides for a method for capturing medical images. The method includes the step of placing a plurality of transducers on a patient body through an enclosure. The plurality of transducers are enabled to capture medical imaging data of a patient. The method further comprises the step of transmitting the medical imaging data of the patient to a remote data processing unit, wherein the remote data processing unit is enabled to enhance the captured medical imaging data of the patient.

### BRIEF DESCRIPTION OF THE DRAWINGS

These and other features, aspects, and advantages of the present invention will be better understood when the following detailed description is read with reference to the accompanying drawings in which like characters represent like parts throughout the drawings, wherein:

FIG. 1 illustrates a block diagram of an medical image capturing apparatus, according to various embodiments of the present invention; and

FIG. 2 is a flowchart illustrating a method for capturing medical images in accordance with various embodiments of the present invention.

### DETAILED DESCRIPTION OF THE INVENTION

The following description is the full and informative description of the best method and system presently contemplated for carrying out the present invention which is known to the inventors at the time of filing the patent application. Of course, many modifications and adaptations will be apparent to those skilled in the relevant arts in view of the following description in view of the accompanying drawings and the appended claims. While the system and method described herein are provided with a certain degree of specificity, the present technique may be implemented with either greater or lesser specificity, depending on the needs of the user. Further, some of the features of the present technique may be used to get an advantage without the corresponding use of other features described in the following paragraphs. As such, the present description should be considered as merely illustrative of the principles of the present technique and not in limitation thereof, since the present technique is defined solely by the claims.

The present invention relates to a system for capturing medical imaging data of a patient and the method thereof. The system includes a plurality of transducers that are placed on an enclosure. The plurality of transducers are configured to capture medical imaging data of a patient when the enclosure is held close to the patient. Further, the system also includes a communication module that transmits the captured medical imaging data to a remote data processing unit. The remote data processing unit is configured to enhance the image captured by the plurality of transducers.

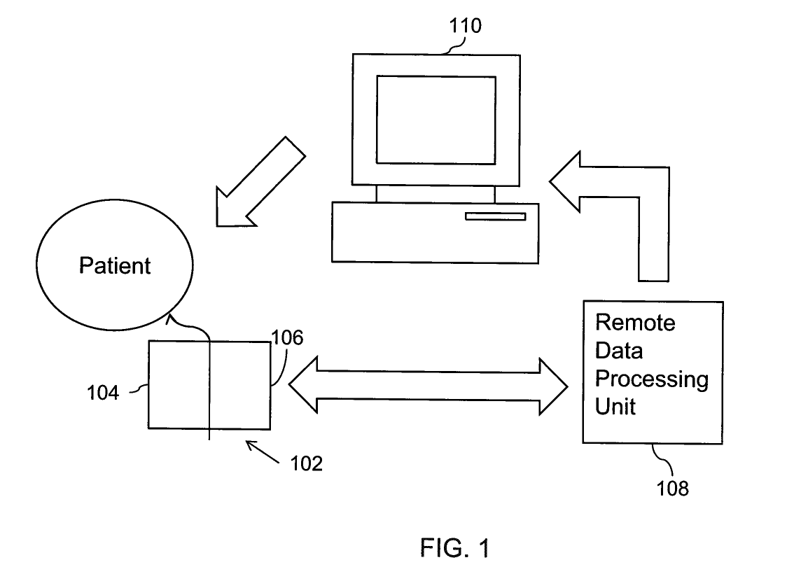

Referring now to the figures, FIG. 1 illustrates a block diagram of a medical image capturing system 100 according to various embodiments of the present invention. The medical image capturing system 100 includes an enclosure 102. The enclosure 102 includes a plurality of transducers 104 and a communication module 106. The plurality of transducers are enabled to capture medical imaging data of a patient. When the enclosure 102 is held close to the patient, the plurality of transducers 104 capture medical imaging data of the patient. Further, the communications module 106 receives this captured medical imaging data from the plurality of transducers 104 and transmits it to a remote data processing unit 108. The remote data processing unit is configured to enhance the captured medical imaging data.

According to one embodiment of the present invention, the plurality of transducers 104 are a plurality of 2-dimensional transducer arrays and the image captured by these transducers is a 2-dimensional ultrasound image. The 2-dimensional transducer array is formed by a system of mini-transducer elements that rotate about an axis perpendicular to the plane of the mini-transducer elements. The communication module 106 transmits this 2-dimensional ultrasound image to the remote data processing unit 108. The remote data processing unit 108 converts this image to a 3-dimensional Doppler image.

According to another embodiment of the present invention, the enclosure 102 can be a mobile phone. The plurality of transducers 104 are placed on the body of the mobile phone in such a way that they can be placed on the patient's body to capture medical imaging data. Further, the enclosure 102 can also be a plastic shell that can hold the plurality of transducers 104. The enclosure 102, according to another embodiment, can be a wearable strap that can be tied around the patient's body. The plurality of transducers 104 can also be placed on a wrist watch that can act as the enclosure 102.

According to another embodiment of the present invention, the communications module 106 is a wireless data transmitter. Further, the communication module 106 can also be a Bluetooth device. The communication module 106 is connected with the plurality of transducers 104 through a data interface. The data interface, in one embodiment, can be a universal serial bus or any other wired or wireless coupling mechanism. The communications module 106 is further capable of transmitting data to the remote data processing unit 108 through a wired or a wireless connection.

The communications module 106, according to yet another embodiment of the present invention, can be the communication system located in a mobile phone. In other embodiments of the present invention, the communication module 106 can utilize a wi-fi connection, a WiMax connection, a GPRS connection, a ZigBee network, Ultrawideband network, or a near field communication network to transmit the medical imaging data captured by the plurality of transducers 104. It may be appreciated by a person skilled in the art that the communication module 106 could use any known communication technique to transmit the medical imaging data.

The remote data processing unit 108 is configured to enhance the medical imaging data captured by the plurality of transducers 104. The process of enhancing can be a heavy computational exercise. The remote data processing unit 108 can use grid computing technology to schedule the task at different connected data processing units to complete the task of enhancing in a faster time. The remote data processing unit 108 can use cloud computing techniques to carry out the task of enhancing the medical imaging data for multiple such image capturing set-ups.

According to another embodiment of the present invention, the remote data processing unit 108 further transmits the enhanced medical imaging data of the patient to a user system 110. The user system 110 can be computer, laptop or a display device associated with a doctor or a hospital laboratory or a caretaker of the patient.

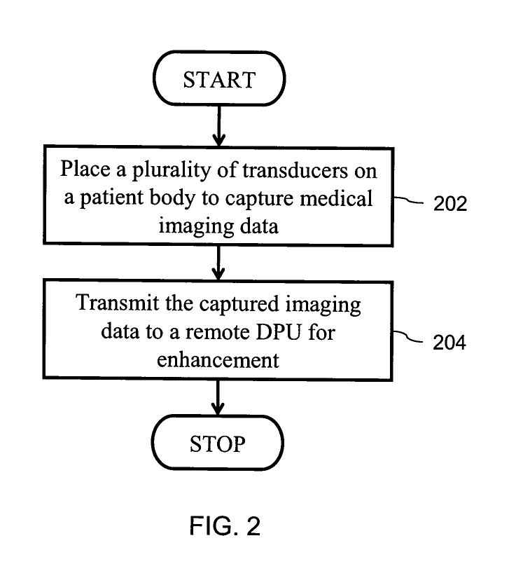

FIG. 2 is a flowchart illustrating a method for capturing medical images in accordance with one embodiment of the present invention. The method for capturing medical images includes a step, 202, of placing a plurality of transducers on a patient body through an enclosure. The plurality of transducers are enabled to capture medical imaging data of the patient. Further, at step 204, the medical imaging data captured by the plurality of transducers is transmitted to a remote data processing unit. The remote data processing unit is configured to enhance the medical imaging data of the patient that is captured by the plurality of transducers.

According to one embodiment of the present invention, the medical imaging data of the patient is transmitted to the remote data processing unit through a communication module. The communication module is communicably coupled with the plurality of transducers.

According to one embodiment of the present invention, the plurality of transducers are a plurality of 2-dimensional transducer arrays and the image captured by these transducers is a 2-dimensional ultrasound image. The 2-dimensional ultrasound image is transmitted to the remote data processing unit through the communication module. The remote data processing unit converts this 2-dimensional image to a 3-dimensional Doppler image.

According to another embodiment of the present invention, the enclosure can be a mobile phone. The plurality of transducers are placed on the body of the mobile phone in such a way that they can be placed on the patient's body to capture medical imaging data. Further, the enclosure can also be a plastic shell that can hold the plurality of transducers. The enclosure, according to another embodiment, can be a wearable strap that can be tied around the patient's body. The plurality of transducers can also be placed on a wrist watch that can act as the enclosure.

According to another embodiment of the present invention, the communications module is a wireless data transmitter. Further, the communication module can also be a Bluetooth device. The communications module, according to yet another embodiment of the present invention, can be the communication system located in a mobile phone. The communication module is connected with the plurality of transducers through a data interface. The data interface, in one embodiment, can be a universal serial bus. The communications module is further capable of transmitting data to the remote data processing unit through a wired or a wireless connection. In other embodiments of the present invention, the communication module can utilize a wi-fi connection, a WiMax connection, a GPRS connection, a ZigBee network, Ultrawideband network, or a near field communication network to transmit the medical imaging data captured by the plurality of transducers. It may be appreciated by a person skilled in the art that the communication module could use any known communication technique to transmit the medical imaging data

The remote data processing unit is configured to enhance the medical imaging data captured by the plurality of transducers. The process of enhancing can be a heavy computational exercise. The remote data processing unit can use grid computing technology to schedule the task at different connected data processing units to complete the task of enhancing in a faster time. The remote data processing unit can use cloud computing techniques to carry out the task of enhancing the medical imaging data.

According to another embodiment of the present invention, the remote data processing unit further transmits the enhanced medical imaging data of the patient to a user system. The user system can be computer, laptop or a display device associated with a doctor or a hospital laboratory or a caretaker of the patient.

As will be appreciated by those ordinary skilled in the art, the foregoing example, demonstrations, and method steps may be implemented by suitable code on a processor base system, such as general purpose or special purpose computer. It should also be noted that different implementations of the present technique may perform some or all the steps described herein in different orders or substantially concurrently, that is, in parallel. Furthermore, the functions may be implemented in a variety of programming languages. Such code, as will be appreciated by those of ordinary skilled in the art, may be stored or adapted for storage in one or more tangible machine readable media, such as on memory chips, local or remote hard disks, optical disks or other media, which may be accessed by a processor based system to execute the stored code. Note that the tangible media may comprise paper or another suitable medium upon which the instructions are printed. For instance, the instructions may be electronically captured via optical scanning of the paper or other medium, then compiled, interpreted or otherwise processed in a suitable manner if necessary, and then stored in a computer memory.

While, the following description is presented to enable a person of ordinary skill in the art to make and use the invention and is provided in the context of the requirement for a obtaining a patent. The present description is the best presently-contemplated method for carrying out the present invention. Various modifications to the preferred embodiment will be readily apparent to those skilled in the art and the generic principles of the present invention may be applied to other embodiments, and some features of the present invention may be used without the corresponding use of other features. Accordingly, the present invention is not intended to be limited to the embodiment shown but is to be accorded the widest cope consistent with the principles and features described herein.

Many modifications of the present invention will be apparent to those skilled in the arts to which the present invention applies. Further, it may be desirable to use some of the features of the present invention without the corresponding use of other features.

Accordingly, the foregoing description of the present invention should be considered as merely illustrative of the principles of the present invention and not in limitation thereof.

What is claimed is:

1. A portable medical image capturing apparatus, comprising:
a plurality of transducers placed on an enclosure, wherein the plurality of transducers are enabled to capture medical imaging data of a patient when the enclosure is held close to the patient; and
a communications module to transmit the medical imaging data of the patient to a remote data processing unit, wherein the remote data processing unit is enabled to enhance the captured medical imaging data of the patient.

2. The portable medical image capturing apparatus as recited in claim 1 wherein the remote server is further configured to transmit the enhanced medical imaging data of the patient to a user system.

3. The portable medical image capturing apparatus as recited in claim 1 wherein the plurality of transducers capture 2-dimensional ultrasound images of the patient.

4. The portable medical image capturing apparatus as recited in claim 3 wherein the remote data processing unit enhances the 2-dimensional ultrasound images of the patient to a 3-dimensional Doppler image.

5. The portable medical image capturing apparatus as recited in claim 1, wherein the communications module is a wireless data transmitter.

6. The portable medical image capturing apparatus as recited in claim 1, wherein the communications module is a Bluetooth device that transmits the medical imaging data to the patient's computer for transmission to the remote server.

7. The portable medical image capturing apparatus as recited in claim 1, wherein the enclosure is a mobile phone.

8. The portable medical image capturing apparatus as recited in claim 1, wherein the enclosure is a plastic shell.

9. The portable medical image capturing apparatus as recited in claim 8, wherein the enclosure is connected to the communications module through a data interface.

10. The portable medical image capturing apparatus as recited in claim 9, wherein the data interface is a universal serial bus.

11. A method for capturing medical images, the method comprising:
placing a plurality of transducers on a patient body through an enclosure, wherein the plurality of transducers are enabled to capture medical imaging data of a patient; and
transmitting the medical imaging data of the patient to a remote data processing unit, wherein the remote data processing unit is enabled to enhance the captured medical imaging data of the patient.

12. The method as recited in claim 11 wherein the medical imaging data of the patient is transmitted to a remote data processing unit through a communication module, wherein the communication module is coupled with the plurality of transducers.

13. The method as recited in claim 11 further comprises transmitting the enhanced medical imaging data of the patient to a user system.

14. The method as recited in claim 11 wherein the plurality of transducers capture 2-dimensional ultrasound images of the patient.

15. The method as recited in claim 14 wherein the remote data processing unit enhances the 2-dimensional ultrasound images of the patient to a 3-dimensional Doppler image.

16. The method as recited in claim 12, wherein the communications module is a wireless data transmitter.

17. The method as recited in claim 16, wherein the communications module is a Bluetooth device that transmits the medical imaging data to the patient's computer for transmission to the remote server.

18. The method as recited in claim 11, wherein the enclosure is a mobile phone.

19. The method as recited in claim 11, wherein the enclosure is a plastic shell.

20. The method as recited in claim 12 further comprises connecting the enclosure with the communication module through a data interface.

21. The method as recited in claim 20, wherein the data interface is a universal serial bus.
